(19) United States
(12) Patent Application Publication
(10) Pub. No.: US 2015/0044650 A1
(43) Pub. Date: Feb. 12, 2015

(54) POSITIVE REINFORCEMENT MESSAGES TO USERS BASED ON ANALYTICS OF PRIOR PHYSIOLOGICAL MEASUREMENTS

(71) Applicant: Cilag GmbH International, Zug (CH)

(72) Inventors: Virendra Parlikar, Fremont, CA (US); Victoria Swenson, San Jose, CA (US); Gregory C. Silvesti, Newtown, PA (US); Miya Osaki, Brooklyn, NY (US); Frances Wilson Howell, Chester Springs, PA (US); Todd Krombholz, Jacksonville, FL (US); Laurence B. Katz, Newtown, PA (US)

(21) Appl. No.: 14/386,848

(22) PCT Filed: Mar. 14, 2013
(86) PCT No.: PCT/US13/31172

§ 371 Date: Sep. 22, 2014

Related U.S. Application Data
(60) Provisional application No. 61/614,931, filed on Mar. 23, 2012.

(51) Int. Cl. G09B 5/00; G06F 19/00; G01N 33/66; G09B 19/00

(52) U.S. Cl. CPC G09B 5/00; G09B 19/00; G06F 19/3406; G01N 33/66
USPC 434/236

(57) ABSTRACT

Described herein are systems and methods to utilize factual information based on stored analyte or physiological data to allow for positive reinforcement of behaviors that are conducive to managing a chronic disease.

### PRIORITY

This application claims the benefits of priority of prior filed U.S. Provisional Patent Application Ser. No. 61/614,931 (Attorney Docket No. LFS5224USPSP with EFS ID 12383246 and Confirmation No. 8511) filed on Mar. 23, 2012, which application is hereby incorporated by reference as if fully set forth herein.

### BACKGROUND

Glucose monitoring is a fact of life for people with diabetes. The accuracy of such monitoring can significantly affect the health and ultimately the quality of life of the person with diabetes. A person with diabetes may measure glucose levels several times a day as a part of the diabetes self management process to ensure glycemic control of the blood glucose within a target range. Failure to maintain target glycemic control can result in serious diabetes-related complications, including cardiovascular disease, kidney disease, nerve damage and blindness. To assist persons with diabetes, there are a number of electronic devices currently available which enable an individual to check the glucose level in a small sample of blood. One such glucose meter is the OneTouch® Verio™ glucose meter, a product which is manufactured by LifeScan.

In addition to glucose monitoring, people with diabetes often have to maintain tight control over their lifestyle, so that they are not adversely affected by, for example, irregular food consumption or exercise. In addition, a health care professional (HCP) dealing with a particular person with diabetes may require detailed information on the lifestyle of the individual to provide effective treatment or modification of treatment for managing diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their HCP on each visit.

The aforementioned methods of recording lifestyle information are inherently difficult, time consuming, and possibly inaccurate. Paper logbooks are not necessarily always carried by an individual and may not be accurately completed when required. Such paper logbooks are small and it is therefore difficult to enter detailed information requiring detailed descriptors of lifestyle events. Furthermore, an individual may often forget key facts about their lifestyle when questioned by a HCP who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system, requires a laborious transcription of information, including lifestyle data, into this secondary data storage. Difficulty of data recordation encourages retrospective entry of pertinent information that results in inaccurate and incomplete records.

There currently exist a number of portable electronic devices that can measure glucose levels in an individual and store the levels for recalling or uploading to another computer for analysis. One such device is the Accu-Check™ Complete™ System from Roche Diagnostics, which provides limited functionality for storing lifestyle data. However, the Accu-Check™ Complete™ System only permits a limited selection of lifestyle variables to be stored in a meter. There is no intelligent feedback from values previously entered into the meter and the user interface is believed to be counterintuitive for an infrequent user of the meter. While it is known to provide messages to the users, such as, for example, US Patent Application Publication No. 2010/0095229, these messages are believed to be less than rigorous in that little or no analysis, i.e., “intelligence” is used to support these messages.

### SUMMARY OF THE DISCLOSURE

Applicants have recognized that, as a supplier of analyte measurement tools for chronic disease such as diabetes management, the tools provided to users must be made to “catch patients doing something right” in the patient's management of a chronic disease with rigorous analysis of the user's glucose measurements. In other words, the tools should be providing patients with messages to reinforce behaviors that lead to the patient's analyte results indicative of good control of disease and which is achieved by the patient consistently over time using rigorous analytics of the measurements and not just any general message. It is believed that much of what patients associate with chronic disease management via physiological measurements (e.g., glucose, cholesterol, peak flow, spirometry, blood pressure, or other physiological indicators) is negative to the patients. Consequently, applicants have realized the need to bring features based on schedules of positive reinforcement and more motivating element to users with a chronic disease but which are based on rigorous analysis of short and long term measurements.

In one aspect, applicants have identified that when in-range results for patients were achieved a major percentage of the time in a relatively short amount of time, a reinforcing message was needed, which was not provided by the existing measurement tools. This recognition by applicants led to the development (by applicants) of specific messages that reinforced positive aspects of the user's analyte testing regimen. Applicants have identified that certain forms of messages were preferred over a simple message of results being in the predetermined range over a predetermined duration spanning multiple days. In annunciating these messages to users, a specific duration was incorporated into the analysis of the user's prior analyte measurements as it was believed that anything less than this specific duration was perceived as “too much” information and detracted from the intrinsic value of the message when received by patients or users.

In another aspect, applicants have also recognized that in order to draw users' attention to times in which the users had done something right to get back into range, and reinforce that behavior, the message should appear after a predetermined number of above range results where the most recent result is in the predetermined range. It was also determined that a preselected number of results was determined to be the optimal number as it was believed that anything more could overwhelm the patients (and reduce the motivational power of the message).

As such, applicants have devised a method of notifying users of physiological trend with a chronic disease management unit to assist persons with diabetes. The disease management unit includes a microprocessor coupled to a memory. The method can be achieved by: measuring with the microprocessor, a most recent analyte measurement of a user by insertion of a glucose test strip into a test strip port of the management unit and deposition of a drop of blood onto the glucose test strip; checking on whether the most recent analyte measurement from the measuring step is within a predetermined range and in the event the most recent analyte measurement is within the predetermined range; conducting for consistency of analyte measurements by: assessing for an absence of consistency message annunciated in prior D number of days, determining whether at least one or more analyte measurements made for each of Z number of separate days within the period of prior D days; obtaining a number of analyte measurements of the plurality of stored analyte measurements for D days that is within the range; calculating whether the number is greater than a predetermined value; and if the assessing, determining and calculating steps are affirmative, annunciating a message to the user of a result of the consistency analysis in which the most recent analyte and the prior analyte measurements over a time duration have been consistently in the predetermined range; or evaluating for progressivity of analyte measurements by: querying as to whether there at least P consecutive prior glucose measurements above range or below range; and if the querying step indicates that at least P consecutive glucose measurements have been stored, annunciating a message that progress has been made in which the most recent glucose measurement is back in the range after being outside the range over one of a time duration or after at least P consecutive prior glucose measurements.

In a variation of this aforementioned method, the first threshold may be about 50 milligrams of glucose per deciliter of blood and the second threshold may be about two to four times that of the first threshold. Moreover, the value for each of D and Z is any number including 1 or more, and preferably D is about 7 and Z is about 3.

In another variation of this aforementioned method, the checking step further includes the steps of: storing in the memory, a plurality of analyte measurements measured prior to the most recent analyte measurement in the measuring step and relating each of the analyte measurements with a time and date at which each of the measurements was taken; if the most recent analyte measurement is one of a value generally equal to or above the second threshold then evaluating for a high trend; and if the most recent analyte measurement is one of a value generally equal to or below the first threshold then evaluating for a low trend.

In another variation of this method, the evaluating for a high trend may include the steps of: defining a referential time interval N that includes a start time before or at generally the same as a time point at which the most recent glucose measurement was taken and an end time after or at generally the same as the time point of the day of the most recent measurement; applying the referential time interval of N to each of prior D days so that the referential time interval N brackets a time point for each prior day that is generally the same as the time point of the most recent glucose measurement; determining whether Y number of prior BG measurements falls within the referential time interval N as applied to each of the prior D days; assessing whether each of the at least X number of prior BG measurements is of a value that is generally equal to or above the second threshold; and if the assessing step indicates that each of at least X glucose measurement is of a value generally equal to or higher than the second threshold, storing a first flag indicative of an above range trend for the duration of D days. The value for each of D and X is any number including 1 and greater, where preferably D is about 7 and X is about 3.

In a variation of this method, the evaluating step for a low trend may include defining a referential time interval N that includes a start time before or at generally the same as a time point at which the most recent glucose measurement was taken and an end time after or at generally the same as the time point of the day of the most recent measurement; applying the referential time interval of N to each of prior D days so that the referential time interval N brackets a time point for each prior day that is generally the same as the time point of the most recent glucose measurement; determining whether Y number of prior BG measurements falls within the referential time interval N as applied to each of the prior D days; assessing whether each of the at least Y number of prior BG measurements is one of a value generally equal to or below the first threshold; and if the assessing step indicates that at least Y glucose measurement is at or lower than the first threshold, storing a second flag indicative of a below range trend for the duration of D days. The value for each of D and X is any number including 1 or more, and preferably D is about 7 and X is about 3.

Further, applicants have also devised a method of notifying users of analyte trend with a diabetes management unit. The unit includes a microprocessor coupled to a memory. The method can be achieved by: measuring with the microprocessor, a most recent analyte measurement of a user; determining whether the most recent analyte measurement from the measuring step is within a range; conducting, in the event the most recent analyte measurement is within the predetermined range, at least one of a consistency analysis and progressivity analysis for prior analyte measurements and the most recent analyte measurement; and annunciating one of (a) a message to the user of a result of the consistency analysis in which the most recent analyte and the prior analyte measurements over a time duration have been consistently in the range or (b) a message to the user of a result of the progressivity analysis in which the most recent analyte measurement is back in the range after being outside the range over one of a time duration or after at least P consecutive prior glucose measurements.

In the methods set forth above, the measuring step may include inserting a glucose test strip into a test strip port of the diabetes management unit and depositing a drop of blood onto the glucose test strip. Alternatively, the determining step may include comparing the most recent analyte measurement with a first threshold and a second threshold of the range. In particular, the comparing step may further include the steps of: storing in the memory, a plurality of analyte measurements measured prior to the most recent analyte measurement in the measuring step and relating each of the analyte measurements with a time and date at which each of the measurements was taken; if the most recent analyte measurement is one of a value generally equal to or above the second threshold then evaluating for a high trend; and if the most recent analyte measurement is one of a value generally equal to or below the first threshold then evaluating for a low trend.

In the aforementioned methods, the evaluating step for a high trend may include the steps of: assessing whether at least one analyte measurement, of the plurality of analyte measurements performed on previous D days within a time frame of N hours before and after a time of the day of the most recent analyte measurement, is at or higher than the second threshold; and if the assessing step indicates that at least one analyte measurement is higher than the second threshold, storing a first flag indicative of an above range trend for the duration of D days.

In the aforementioned methods, the evaluating for a low trend may include:

evaluating whether at least one analyte measurement, of the plurality analyte measurements performed on previous D days within a time frame of N hours before and after a time of the day of the most recent analyte measurement, is at or lower than the first threshold; and if the evaluating indicates that at least one analyte measurement is lower than the first threshold, storing a second flag indicative of a below range trend for the duration of D days.

In the previously mentioned methods, the conducting may also include evaluating for consistency of analyte measurements by: assessing for an absence of consistency message annunciated in prior D number of days; determining whether at least one or more analyte measurements made for each of Z number of days within the period of prior D days; obtaining a number of analyte measurements of the plurality of stored analyte measurements for D days that is within the range; calculating whether the number is greater than a predetermined value; and if the assessing, determining and calculating steps are affirmative, annunciating a message to the user of a result of the consistency analysis in which the most recent analyte and the prior analyte measurements over a time duration have been consistently in the predetermined range. The value for each of D and Z is any number including 1 or more, and preferably D is about 6 and Z is about 3.

In such methods described above, the conducting step may include evaluating for progressivity of analyte measurements by: evaluating whether the most recent analyte measurement is within the predetermined range; determining whether at least one of an above range flag or below range flag has been stored for the period of D days; and annunciating a message that progress has been made in the event the determining step indicates that at least one of the above range flag and below range flag has been stored and the evaluating reflects that the most recent analyte measurement is within the predetermined range defined by the low and high thresholds. And in the event that the assessing, determining, and calculating steps are negative or the evaluating, determining, and annunciating steps are negative, annunciating a message to continue with analyte measurements.

Applicants have also devised a chronic disease management system that provides users with reinforcing messages to manage the chronic disease of users. The system includes a biosensor unit that provides physiological measurement data of a user; and a chronic disease management unit that includes a microprocessor and memory. The microprocessor is in communication with the biosensor unit to receive a plurality of physiological measurements reflective of a health condition of the user. The microprocessor is also coupled to a memory and configured to: store the plurality of physiological measurements as collected from the biosensor; determine whether a most recent physiological measurement is within a predetermined range; evaluate (a) whether the plurality of physiological measurements including the most recent physiological measurement are consistently within the predetermined range over a time duration or (b) whether the most recent physiological measurement is within the predetermined range while prior plurality of physiological measurements have been out of the predetermined range during a time duration; and annunciate a message indicating a result of evaluation (a) or (b) to the user.

In this system, the message of the result of the evaluation for consistency analysis may include at least one of: (1) indication that a percentage of physiological measurements including the most recent physiological measurement in the last D days are in the predetermined range; (2) indication that a number out of a total number of physiological measurements including the most recent measurement in the last D days are in the predetermined range; or (3) indication that the user is doing well by having a percentage of the physiological measurements including the most recent physiological measurement in the last D days are in the predetermined range.

Also in this system, the message of the result of the evaluation for progressivity analysis may include at least one of an: (1) indication that the most recent physiological measurement is back in the predetermined range; (2) indication that the most recent physiological measurement is back in the predetermined range after being a number of times out of the predetermined range; or (3) indication that the user is back in the predetermined range after being out of the predetermined range.

In this system, the evaluation by the microprocessor for consistency of physiological measurements is achieved by: an assessment for an absence of consistency message annunciated in prior D number of days, a determination of whether at least one or more physiological measurements made for each of Z number of days within the period of prior D days; an estimation of a number of physiological measurements of the plurality of stored physiological measurements for D days that is within the range; a calculation of whether the number is greater than a predetermined value; and if the assessment, determination and calculation by the processor are affirmative, the processor is configured to annunciate a message to the user of a result of the consistency analysis in which the most recent physiological measurement and the prior physiological measurements over a time duration have been consistently in the predetermined range.

Alternatively, the evaluation by the microprocessor for progressivity of physiological measurements is by: an evaluation of whether the most recent physiological measurement is within the predetermined range; a determination of whether at least one of an above range flag or below range flag has been stored for the period of D days; and the processor is configured to annunciate a message that progress has been made by the user in the event the microprocessor determines that at least one of the above range flag and below range flag has been stored and the evaluation by the microprocessor reflects that the most recent physiological measurement is within the predetermined range. The value for each of D, N, Y, or X is any number including 1 or more, and preferably D is about 4, N is about 3, Y is about 1 and X is about 2.

In the aforementioned aspects of the disclosure, the steps of determining, estimating, calculating, computing, deriving and/or utilizing (possibly in conjunction with an equation) may be performed by an electronic circuit or a processor. These steps may also be implemented as executable instructions stored on a computer readable medium; the instructions, when executed by a computer may perform the steps of any one of the aforementioned methods.

In additional aspects of the disclosure, there are computer readable media, each medium comprising executable instructions, which, when executed by a computer, perform the steps of any one of the aforementioned methods.

In additional aspects of the disclosure, there are devices, such as test meters or analyte testing devices, each device or meter comprising an electronic circuit or processor configured to perform the steps of any one of the aforementioned methods.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE FIGURES

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A illustrates a chronic disease management system that includes an analyte measurement and data management unit and a biosensor.

FIG. 1B illustrates, in simplified schematic, an exemplary circuit board of a chronic disease data management unit.

FIG. 2 illustrates an overview of a process flow for a user interface of the chronic disease data management unit.

FIG. 3 illustrates a routine to determine if a trend of the analyte measurements is indicative of an above range trend for storage of a flag to the same.

FIG. 4 illustrates a routine to determine if a trend of the analyte measurements is indicative of a below range trend for storage of a flag to the same.

FIGS. 5A, 5B, 5C, 5D, and 5E illustrate an example of the application of the Referential Time Interval or sliding time window about the time point of the most recent analyte measurement as applied to measurements made in prior days at the same time point as the most recent measurement.

FIG. 6 illustrates consistency analysis routine for use by the main routine.

FIG. 7 illustrates a progressivity analysis routine for use by the main routine.

FIG. 8 illustrates various devices and systems in which the invention described and illustrated herein may be utilized.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment.

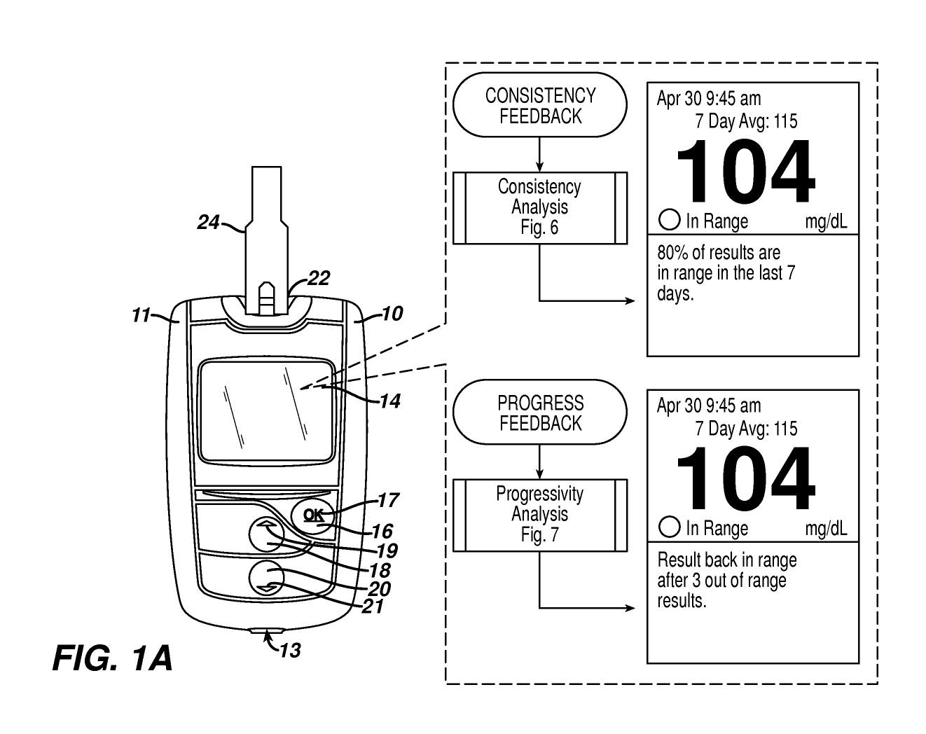

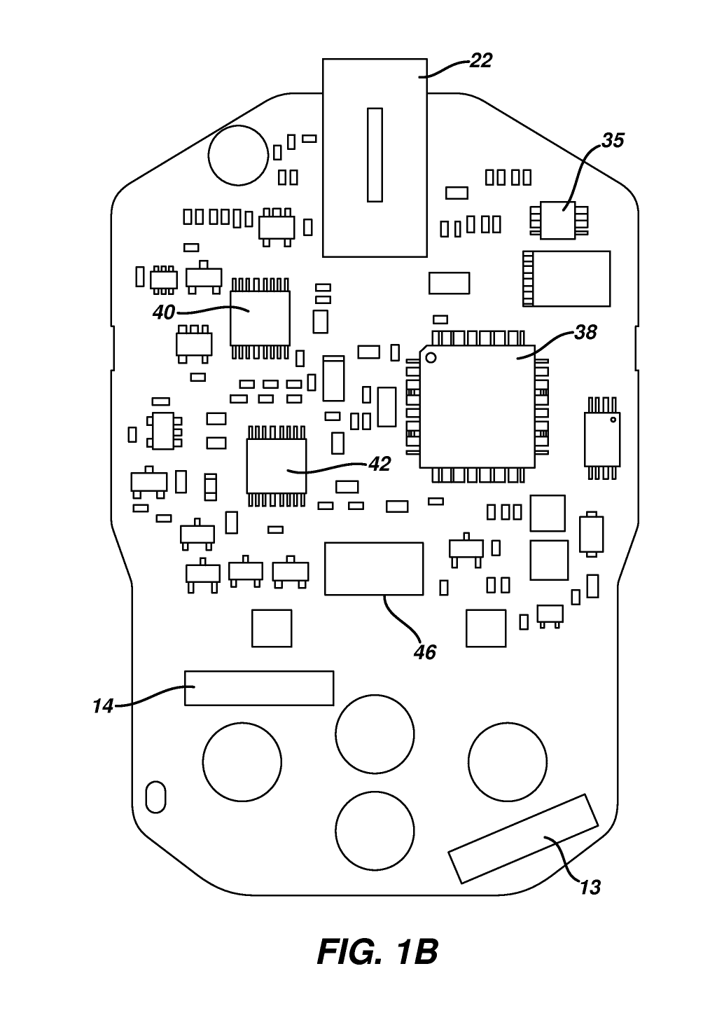

FIG. 1A illustrates a chronic disease management system that includes a data management unit 10 (“DMU”) and a biosensor in the form of a glucose test strip 24. It is noted that while the biosensor is shown in the form of a test strip to test blood glucose, a continuous glucose monitor can also be utilized as an alternative to the embodiments described herein.

Analyte meter or DMU 10 can include a housing 11, user interface buttons (16, 18, and 20), a display 14, a strip port connector 22, and a data port 13, as illustrated in FIG. 1A. User interface buttons (16, 18, and 20) can be configured to allow the entry of data, navigation of menus, and execution of commands. Data can include values representative of analyte concentration, and/or information, which are related to the everyday lifestyle of an individual. Information, which is related to the everyday lifestyle, can include food intake, medication use, occurrence of health check-ups, and general health condition and exercise levels of an individual. Specifically, user interface buttons (16, 18, and 20) include a first user interface button 16, a second user interface button 18, and a third user interface button 20. User interface buttons (16, 18, and 20) include a first marking 17, a second marking 19, and a third marking 21, respectively, which allow a user to navigate through the user interface. Although the buttons are shown as mechanical switches, a touch screen interface with virtual buttons may also be utilized. As represented in FIG. 1A, the DMU is provided with various user-interfaces including the user interface UI to provide for consistency or progressivity feedback to the user's analyte measurements over time.

The electronic components of meter 10 can be disposed on a circuit board 34 that is within housing 11. FIG. 1B illustrates (in simplified schematic form) the electronic components disposed on a top surface of circuit board 34. On the top surface, the electronic components include a strip port connector 22, an operational amplifier circuit 35, a microcontroller 38, a display connector 14a, a non-volatile memory 40, a clock 42, and a first wireless module 46. On the bottom surface, the electronic components may include a battery connector (not shown) and a data port 13. Microcontroller 38 can be electrically connected to strip port connector 22, operational amplifier circuit 35, first wireless module 46, display 14, non-volatile memory 40, clock 42, battery, data port 13, and user interface buttons (16, 18, and 20).

Operational amplifier circuit 35 can include two or more operational amplifiers configured to provide a portion of the potentiostat function and the current measurement function. The potentiostat function can refer to the application of a test voltage between at least two electrodes of a test strip. The current function can refer to the measurement of a test current resulting from the applied test voltage. The current measurement may be performed with a current-to-voltage converter. Microcontroller 38 can be in the form of a mixed signal microprocessor (MSP) such as, for example, the Texas Instrument MSP 430. The TI-MSP 430 can be configured to also perform a portion of the potentiostat function and the current measurement function. In addition, the MSP 430 can also include volatile and non-volatile memory. In another embodiment, many of the electronic components can be integrated with the microcontroller in the form of an application specific integrated circuit (ASIC).

Strip port connector 22 can be configured to form an electrical connection to the test strip. Display connector 14a can be configured to attach to display 14. Display 14 can be in the form of a liquid crystal display for reporting measured analyte levels, and for facilitating entry of lifestyle related information. Display 14 can optionally include a backlight. Data port 13 can accept a suitable connector attached to a connecting lead, thereby allowing glucose meter 10 to be linked to an external device such as a personal computer. Data port 13 can be any port that allows for transmission of data such as, for example, a serial, USB, or a parallel port. Clock 42 can be configured to keep current time related to the geographic region in which the user is located and also for measuring time. The DMU can be configured to be electrically connected to a power supply such as, for example, a battery.

Referring back to FIG. 1A, analyte test strip 24 can be in the form of an electrochemical glucose test strip. Test strip 24 can include one or more working electrodes and a counter electrode. Test strip 24 can also include a plurality of electrical contact pads, where each electrode can be in electrical communication with at least one electrical contact pad. Strip port connector 22 can be configured to electrically interface to the electrical contact pads and form electrical communication with the electrodes. Test strip 24 can include a reagent layer that is disposed over at least one electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). An exemplary mediator suitable for use in the reagent layer includes ferricyanide, which in this case is in the oxidized form. The reagent layer can be configured to physically transform glucose into an enzymatic by-product and in the process generate an amount of reduced mediator (e.g., ferrocyanide) that is proportional to the glucose concentration. The working electrode can then measure a concentration of the reduced mediator in the form of a current. In turn, glucose meter 10 can convert the current magnitude into a glucose concentration. Details of the preferred test strip are provided in U.S. Pat. Nos. 6,179,979; 6,193,873; 6,284,125; 6,413,410; 6,475,372; 6,716,577; 6,749,887; 6,863,801; 6,890,421; 7,045,046; 7,291,256; 7,498,132, all of which are incorporated by reference in their entireties herein with a copy attached to the appendix of this application.

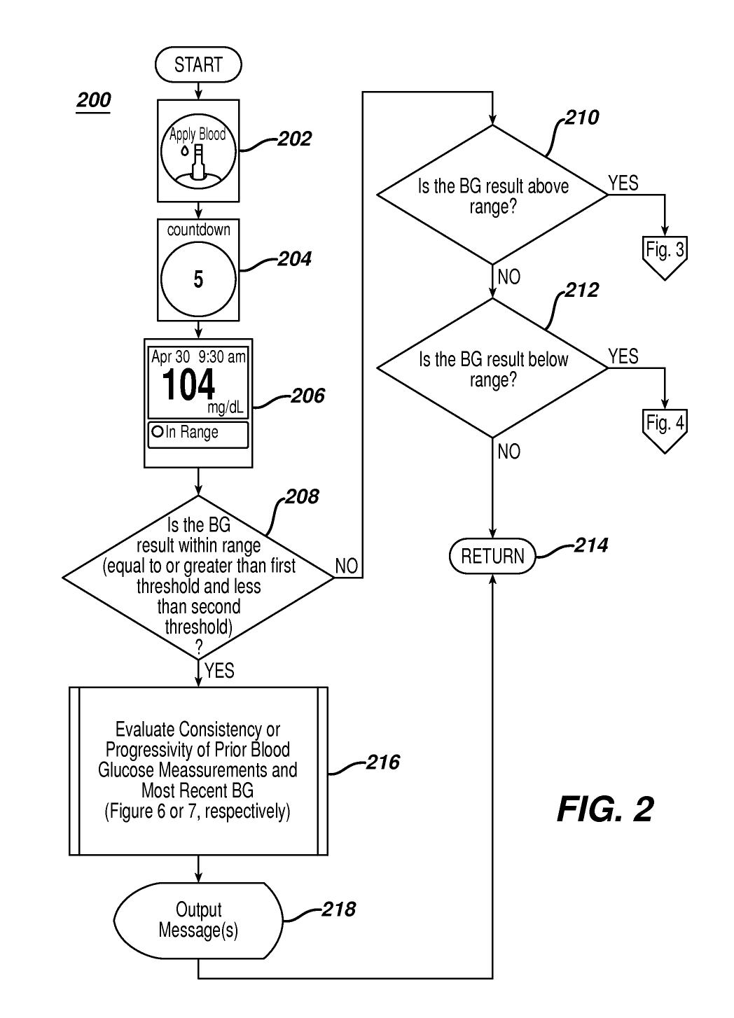

Referring to FIGS. 2-6, an exemplary process flow for portions of the user interface for the DMU is provided. Specifically, in FIG. 2, the process flow begins at step 200 when a suitable test strip 24 is inserted into the DMU 10 (of FIG. 1A). The DMU 10 counts down in step 204. Once countdown is completed, an analyte measurement (which means any biosensor that can determine glucose in any physiological fluid (“BG”)) is annunciated in step 206. As used herein, the term “annunciated” and variations on its root term indicate that an announcement may be provided via text, audio, visual or a combination of all modes or mediums of communication to a user. To inform the user of the qualitative aspect of the result, an indicia 207 can be provided to indicate whether the result is outside of the desired range via a red indicia (or flashing message) or in-range by way of a green indicia or the like. Thereafter, the system, via software and microprocessor, determines in step 208 whether this latest BG result, referenced as “the most recent BG result” is within a predetermined range (or “in-range”) of analyte values. The “in-range” for this embodiment is from about 60 milligrams of glucose per deciliter of blood (“mg/dL”) to about 200 mg/dL with a default low threshold of 70 mg/dL and a default high threshold of 180 mg/dL. However, these thresholds for the range can be changed by the user or HCP to different units or measurements.

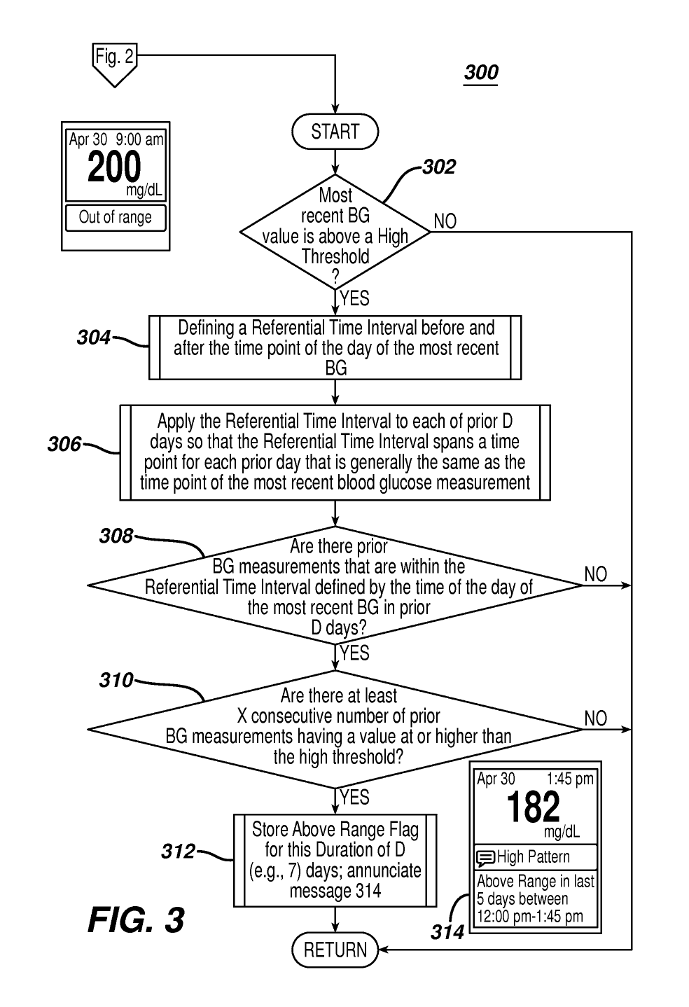

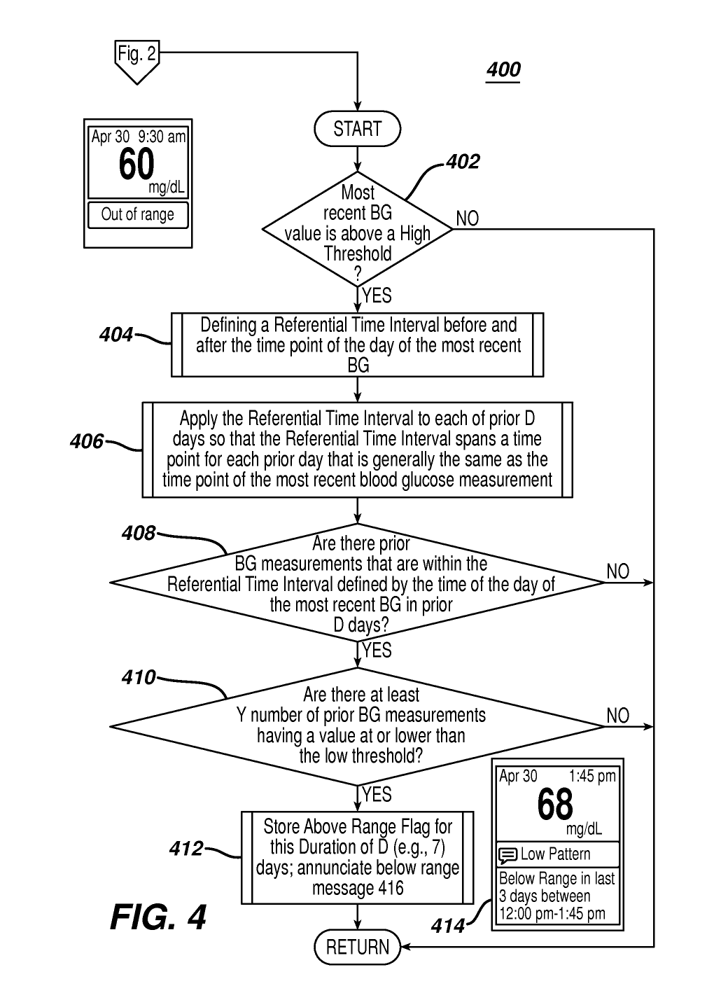

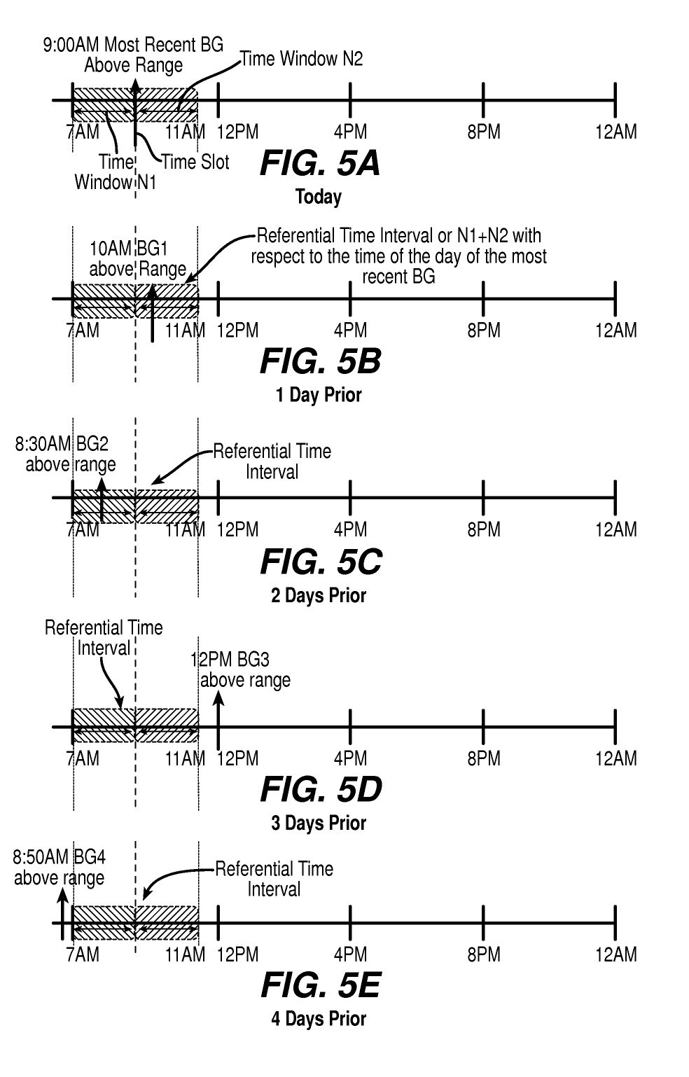

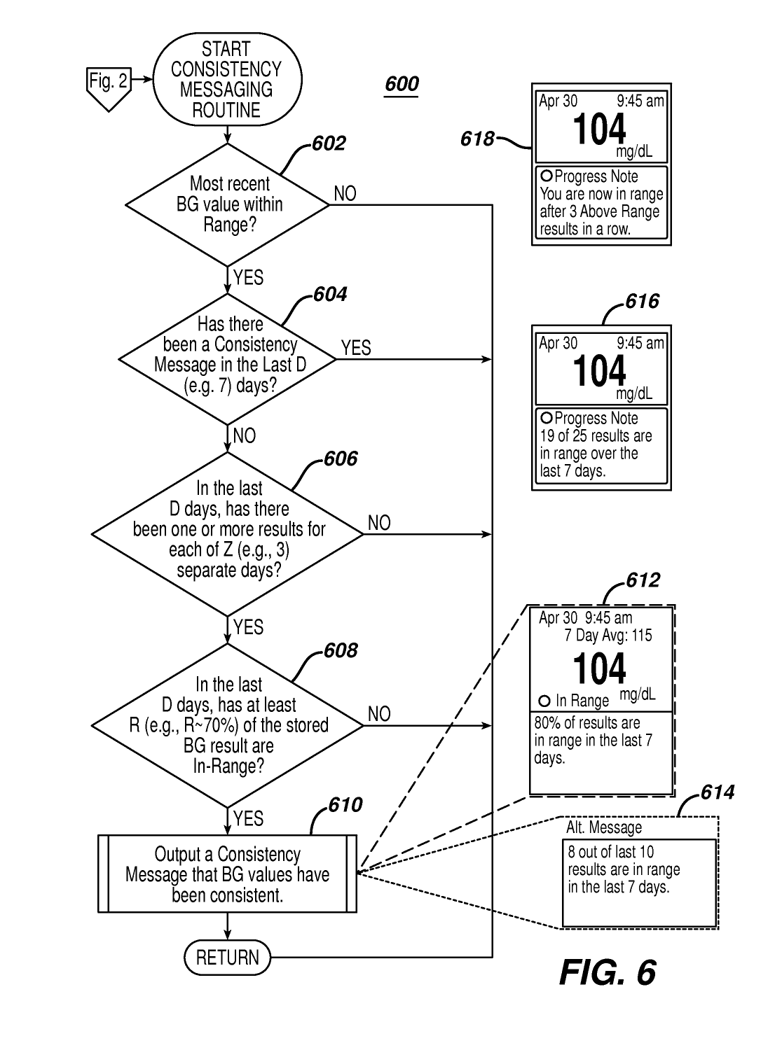

If the system determines at step 208 that the BG result is not in-range, the process moves to steps 210 and 212 so that the system can determine whether the out of the predetermined range result is part of an above range trend or a below range trend. It should be noted that an indication of “in-range” for a measurement value means that the measured value is equal to greater than a low threshold but less than a high threshold. At step 210, if the result of the latest BG is determined as being above the range, the process moves to routine 300 (FIG. 3); and if the latest BG result is below range, the process moves to routine 400 (FIG. 4). On the other hand, if the system determines at step 208 that the BG result is in-range, the process moves to routines provided for in step 210 to determine consistency (FIG. 5) or progressivity (FIG. 6) of the BG results including the latest BG. Thereafter, messages can be annunciated at step 212 of the results from these consistency or progressivity routines.

Referring to FIG. 3, routine 300 for determining an above range trend begins with evaluating step 302 in which a determination is made as to whether a most recent BG result is at or above a high threshold and if true, the system defines a Referential Time Interval (which will be explained further below) before and after a time point in a day of the most recent BG measurement at step 304. At step 306, the system applies the Referential Time Interval of the day of the most recent BG result to each of prior D days so that the referential time interval N brackets a time point for each prior day that is generally the same as the time point for the most recent BG. At step 308, the system queries as to whether there are prior BG measurements in prior D days that were taken during the Referential Time Interval and if step 308 is affirmative, the system queries at step 310 whether there are at least X number of prior BG measurements having values at or higher than the second or high threshold. If the query 310 is affirmative, the system stores a flag indicative of an above range trend in step 312 and separately or concurrently annunciate message 314 that user is above range in the prior D days between start time and end time of the Referential Time Interval. In sum, steps 304-310 are to determine whether on X of the previous D days within an N hour sliding window of the time of the day of the most recent BG measurement, there has been a BG measurement above the high threshold. Preferably, X is set to about 2, D to about 4, and N to about 3.

An explanation of the Referential Time Interval or sliding time window will now be described. For ease of understanding of this “Referential Time Interval” about the time point in a day of the most recent BG measurements, reference is made to FIGS. 5A-5E where it is assumed that the users made a series of measurements over consecutive days. In FIG. 5A, it is assumed that a most recent BG measurement was made today at 9:00 AM in which the most recent BG reading is 200 mg/dL (FIG. 3). This reading is above the range of 70 mg/dL-180 mg/dL and therefore the process flow of FIG. 2 would arrive at step 210 and then onto step 302 of FIG. 3. For the system to determine if there was an above range trend, the system looks over measurements made today and prior D number of days to conduct its above range trend analysis. For brevity, D is set to equal to 4 in this example so that the measurements of 4 days (FIGS. 5B, 5C, 5D, and 5E) are also included with today (FIG. 5A).

Using the time point of the day (e.g., 9:00 AM) for the most recent BG (for today in FIG. 5A), a time window of N1 hours (e.g., where N1=2) before the reference time point of the day (in this example, at 9 AM for the latest BG value) and a time window of N2 hours (e.g., N2=2) are defined as a “Referential Time Interval” (where the interval=N1+N2 or 4 hours total) that brackets the reference time point (at 9 AM) for today. This Referential Time Interval from 7 AM to 11 PM (defined in step 304 of FIG. 3) is then applied (in step 306 of FIG. 3) to each day of prior D days so that the Referential Time Interval N brackets a time point for each of prior day, at which time point for each prior day (at 9 AM) is generally the same as the time point (at 9 AM) of the most recent BG measurement. While the time slot of the day (9 AM) of the most recent BG is preferably set as equitemporal in the Referential Time Interval (e.g., the same number of hours before and after the time slot of the most recent BG), the Referential Time Interval could be configured so that the number of hours before the time slot for the most recent BG is different from the number of hours after the time slot of the most recent BG measurement (e.g., 2 hours before 9 AM and 4 hours after 9 AM).

In this example for FIGS. 3 and 5A-5E, the microprocessor polls for previously stored glucose measurements made in the previous D number of days within a windows of N1+N2 hours bracketing the same time slot in a day (e.g., at 9 AM) in which the most recent BG measurement of 200 mg/dL was made. In step 308, the processor looks for BG measurements made in prior days that would fall within the Referential Time Interval N for each of those prior days. In the example of FIGS. 5A-5E, the Referential Time Interval would overlay the measurement BG1 made at 10 AM one day prior (FIG. 5B). The same Referential Time Interval would also cover measurement BG2 made at 830 AM in FIG. 5C for two-days prior (FIG. 5C). However, the same Referential Time Interval would not include measurement BG3, which was taken at 12 PM three-days prior (FIG. 5D). Similarly, the Referential Time Interval would not cover measurement BG4 (at 750 AM) taken four days (FIG. 5E) prior to today (FIG. 5A). From the evaluation step 308, there are three prior BG measurements (for one day prior (FIG. 5B), two-days prior (FIG. 5C), and three-days prior) that are within the Referential Time Interval N defined by the most recent BG measurement of today. Note that in the examples set forth in FIGS. 5A-5E, only one glucose concentration per day was depicted. In real-life situations, there may be more than one or more glucose concentration per day that are above the high threshold or below the low threshold. In such a case, the number of combinations of BG (i.e., glucose) measurements that need to be evaluated by the logic of the system will increase. Moreover, there could be more than one above range trend or more than one below range trend.

To reduce the number of confusing messages, prioritization of the above-range trend or below-range trend used in the analysis can be based on the following: once a BG value is used for one of an above-range or below-range trend, it will no longer be included in other trend; if multiple trends are detected, the tightest clustering of results will be the one utilized; or if there are multiple high and low BG measurements within an hour, only the first will be included in trend analysis (i.e., if there are either multiple high values with an hour or multiple low values within an hour, only the first will be included in the trend analysis). Alternatively, the prioritization can be based on chronological closeness or based on the tightness of the clustering which can be determined by the closest 2 BG results in time to the most recent BG result, or the closest 3 BG results in time to the most recent BG result.

Referring back to FIG. 3, if step 308 is affirmative, the process moves to step 310. In step 310, if there are at least X number of prior BG measurements having a value at or higher than the high threshold then the process moves to step 312 to store a flag of an above range trend. In other words, from steps 308 and 310, if there are at least X prior glucose measurement(s) greater than the high threshold that falls on prior consecutive D days within a time span defined with respect to the time of the day of the most recent BG measurement, then the evaluation 308 and 310 are deemed as being in the affirmative and the process moves to step 312 in which the processor store a flag, tag or other indicator of an above range trend for the duration of prior D days. The system can also annunciate a message that an above range pattern has been detected. In these embodiments, the preferred value of each of time windows N1 and N2 is set to about 90 minutes for a sliding time window of about 180 minutes (i.e., about 3 hours); the number of consecutive prior measurements X is set to about 2, and the number of prior days D is set to about 6.

In an alternative embodiment, three or more prior glucose measurements equal to greater than the second threshold in the Referential Time Interval over at least a Z number of prior days (where Z^{˜} any integer number including 7, 14, 21, 30, 60) must be met to cause the system to store an above range flag. In other embodiments, the user can select details relating to this flag, which may include, for example, a table of the multiple BG measurements with the corresponding dates and times; a number of time the user has been above range; the particular date and time of these readings; the exact value on which an above range flag was made based and whether the reading was taken with a tag of before meal (indicated by a suitable icon such as, for example, an uneaten fruit, such as, for example, an apple).

Referring back to FIG. 2, if step 212 is indicative of the most recent BG being below range, the system moves to routine 400 (FIG. 4). In FIG. 4, the routine 400 begins with evaluating step 402 in which a determination is made as to whether a most recent BG result is less than a first or low threshold and if true, the system defines a Referential Time Interval before and after the time point in a day of the most recent BG measurement at step 404. In this example, the Referential time Interval could be set to 2 hours before 9 AM (so that the start time is at 7 AM) and 2 hours after 9 AM (so that the end time is at 11 AM). At step 406, the system applies the Referential Time Interval to each of prior D days so that the Referential Time Interval brackets a time point for each prior day that is generally the same as the time point for the most recent BG. At step 408, the system queries as to whether there are prior BG measurements in prior D days that were taken during the Referential Time Interval and if step 408 is affirmative, the system queries at step 410, whether there are at least Y number of prior BG measurements having values at or higher than the high threshold. If the query 410 is affirmative, the system stores a flag indicative of a below range trend in step 412 and separately or concurrently annunciate a message 414 that the user is below range in prior D days between start time and end time of the Referential Time Interval. Thereafter the logic returns to the main routine 416. In sum, steps 404-410 are set up to determine whether on Y of the previous D days within a N hour sliding window of the time of day of the glucose result there has been a glucose result below the a low threshold, and if affirmative, the logic store a below range flag or at the same time annunciate a message that a below range trend has been detected. Preferably, Y is set to about 1, D is set to about 4, and N is set to about 180 minutes.

In an alternative embodiment, three or more prior BG measurements equal to or lower than the first threshold in the Referential Time Interval over at least a Z number of prior days (where Z^{˜} any integer number from 1 to 7, 14, 21, 30, 60) must be met to cause the system to store a below range flag or annunciate a message indicative of a below-range trend being detected.

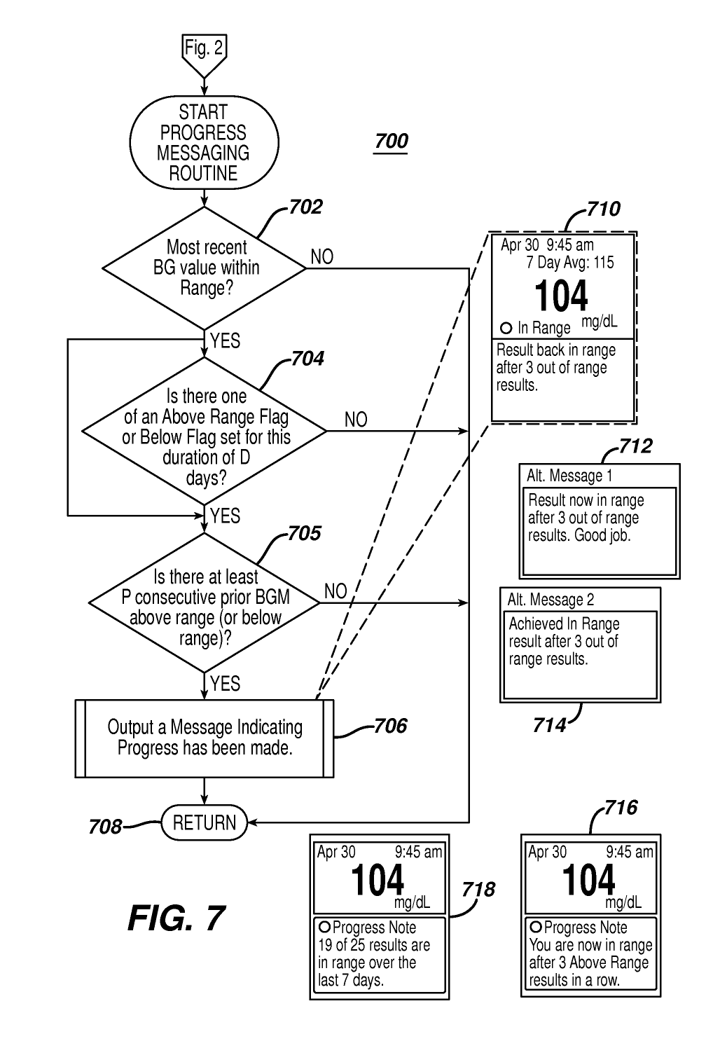

With reference back to FIG. 2, if the system determines that the latest BG is within the predetermined range, the system evaluates for prior and latest BG results for one of consistency or progressivity in the glucose measurements over a period of days. In particular, at step 216, the system flows to FIGS. 6 and 7 to perform these analyses whenever the most recent BG value is in-range.

For consistency analysis, reference is made to FIG. 6 where the routine 600 starts by confirming at step 602 that the most recent BG value is in-range and if true or affirmative, the system logic moves to step 604 in which the system logic checks to see if no consistency message was annunciated in the last D days. If step 604 returns an affirmative or true state then the system moves to step 606, otherwise the logic returns to the main routine. At step 606, the system logic checks for whether in the last D days, on at least Z number of separate days, there has been at least one BG result for each of the Z days. If the query 606 returns a true or affirmative, the system moves to step 608 in which a query is made as to whether in the last D days, at least R number of stored result (or a percentage of the total stored BG results for the D days) is within the predetermined range. If query 608 is true, the system annunciates a message 612 (or alternative messages 614, 616, and 618) at step 610 to inform the user that a percentage (or a number of BG values) is within the predetermined range in the last D days.

For progressivity analysis of analyte measurements, reference is made to FIG. 7 where routine 700 starts by confirming that the most recent BG value is in-range and if the query 702 is true or affirmative, the logic moves to step 704 and 705 (or alternatively from step 702 directly to step 705), otherwise the system logic returns to the main routine in step 708. In step 704, the system logic checks to see if there is one of an above-range flag stored (FIG. 3) or a below-range flag stored (FIG. 4). If query 704 returns a true or affirmative, the logic flows to step 706 which annunciates to the user with message 710 (or alternative messages 712, 714, 716, and 718) that the BG result (i.e., the latest result) is back in-range. In an alternative, if query 704 returns a true result, the logic flows to step 705, in which the system gleans from prior glucose measurements of at least P consecutive (e.g., P=3) glucose results that are above the range (or below the in-range) during the time duration being considered. In a preferred embodiment, if the query in step 702 is true, the logic flows directly to step 705 (bypassing step 704) in order to glean if at least P consecutive results are above the in-range (or below the in-range).

Instead of a single message as in each of FIGS. 6 and 7, there may be at least two (and preferably at least three) differently formatted messages that may be presented to the user in sequence or in a random sequence to reduce the perception of repetition by the user. In particular, these three differently formatted messages are utilized to communicate semantically a similar message. For example in FIG. 6, message template 612 may be provided initially. On different occasions, a different message template 614 with a similar meaning can also be provided. In FIG. 7, message template 710 can be provided initially and thereafter message templates 712 and 714 can also be provided with similar meaning. Message templates 612, 614, 616, 618, 708, 710, 712, 714, 716, 718, and the like may cycle sequentially or randomly so that the user does not perceive the identically formatted message over and over again, which may lulls the user into ignoring the pattern messages being communicated to the user. Alternatively, each message for routines 600 and 700 can be set in a permanent format without any variation.

In the preferred embodiments, the window of X hours includes from about 1 to about 8 hours and the D number of days or the Z number of days may range from about 2 to about 21 days. In another preferred embodiment, the window of X hours include about 3 hours and the D number of days may range from about 2 to about 30 days, and most preferably from about 2 to about 7 days including the day of the most recent glucose measurement.

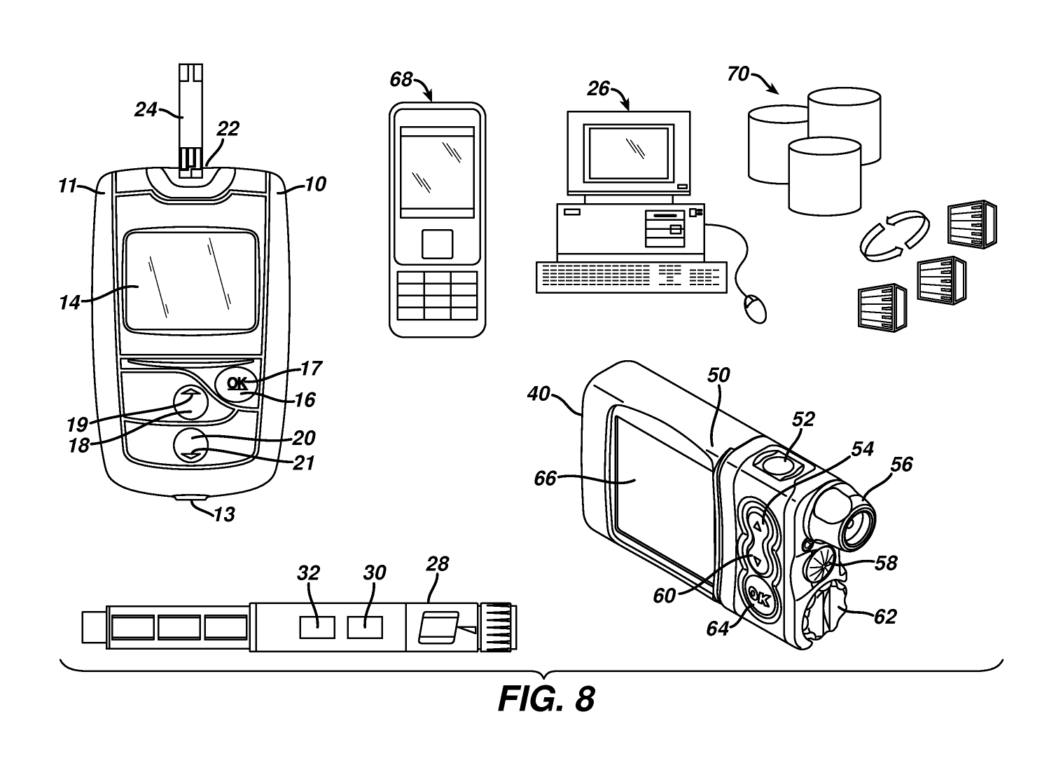

Although exemplary embodiments have been described in relation to a glucose meter, other data management devices may also be utilized. For example, with reference to FIG. 8, analyte measurement and management unit 10 can be configured to wirelessly communicate with a handheld glucose-insulin data management unit or DMU such as, for example, an insulin pen 28, an insulin pump 48, a mobile phone 68, or through a combination of the exemplary handheld glucose-insulin data management unit devices in communication with a personal computer 26 or network server 70, as described herein. As used herein, the nomenclature “DMU” represents either individual unit 10, 28, 48, 68, separately or all of the handheld glucose-insulin data management units (28, 48, 68) usable together in a disease management system. Further, the analyte measurement and management unit or DMU 10 is intended to include a glucose meter, a meter, an analyte measurement device, an insulin delivery device or a combination of or an analyte testing and drug delivery device. In an embodiment, analyte measurement and management unit 10 may be connected to personal computer 26 with a cable. In an alternative, the DMU may be connected to the computer 26 or server 70 via a suitable wireless technology such as, for example, GSM, CDMA, BlueTooth, WiFi and the like.

Referring to FIG. 8, it should be noted that an insulin pen can be utilized to perform as described herein. Such insulin pen 28 may be provided with an electronic module 30 programmed to carry out the exemplary methods and variations thereof to assist user in management of diabetes. The device 28 may include a wireless module 32 disposed in the housing that, automatically without prompting from a user, transmits a signal to a wireless module 46 of the DMU 10. The wireless signal can include, in an exemplary embodiment, data to (a) type of therapeutic agent delivered; (b) amount of therapeutic agent delivered to the user; (c) time and date of therapeutic agent delivery; or (d) trends of high or low BG results. A non-limiting example of such a user-activated therapeutic agent delivery device is described in co-pending U.S. Non-Provisional application Ser. No. 12/407,173 (tentatively identified by Attorney Docket No. LFS-5180USNP); Ser. No. 12/417,875 (tentatively identified by Attorney Docket No. LFS-5183USNP); and Ser. No. 12/540,217 (tentatively identified by Attorney Docket No. DDI-5176USNP), which is hereby incorporated in whole by reference hereto this application. Another non-limiting example of such a user-activated therapeutic agent delivery device is an insulin pen 28. Insulin pens can be loaded with a vial or cartridge of insulin, and can be attached to a disposable needle. Portions of the insulin pen can be reusable, or the insulin pen can be completely disposable. Insulin pens are commercially available from companies such as Novo Nordisk, Aventis, and Eli Lilly, and can be used with a variety of insulin, such as Novolog, Humalog, Levemir, and Lantus.

In yet a further alternative to the glucose meter 10, as shown in FIG. 8, a therapeutic dosing device can also be a pump 48 that includes a housing 50, a backlight button 52, an up button 54, a cartridge cap 56, a bolus button 58, a down button 60, a battery cap 62, an OK button 64, and a display 66. Pump 48 can be configured to dispense medication such as, for example, insulin for regulating glucose levels. As noted earlier, a microprocessor can be programmed to generally carry out the steps of various processes described herein. The microprocessor can be part of a particular device, such as, for example, a glucose meter, an insulin pen, an insulin pump, a server, a mobile phone, personal computer, or mobile hand held device.

Furthermore, the various methods described herein can be used to generate software codes using off-the-shelf software development tools such as, for example, Visual Studio 6.0, C or C++ (and its variants), Windows 2000 Server, and SQL Server 2000. The methods, however, may be transformed into other software languages depending on the requirements and the availability of new software languages for coding the methods. Additionally, the various methods described, once transformed into suitable software codes, may be embodied in any computer-readable storage medium that, when executed by a suitable microprocessor or computer, are operable to carry out the steps described in these methods along with any other necessary steps.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

1. A method of notifying users of glucose trends with a diabetes management unit having a microprocessor coupled to a memory, the method comprising:
measuring with the microprocessor, a most recent glucose measurement of a user by insertion a glucose test strip into a test strip port of the diabetes management unit and deposition of a drop of blood onto the glucose test strip;
checking on whether the most recent glucose measurement from the measuring step is within a predetermined range;
in the event the most recent glucose measurement is within the predetermined range, conducting for consistency of prior glucose measurements by:
assessing for an absence of consistency message annunciated in prior D number of days,
determining whether at least one or more glucose measurements were made for each of Z number of days within the period of prior D days;
obtaining a number of glucose measurements of the plurality of stored glucose measurements for D days that is within the range;
calculating whether the number is greater than a predetermined value; and
if the assessing, determining and calculating steps are affirmative, annunciating a message to the user of a result of the consistency analysis in which the most recent glucose and the prior glucose measurements over a time duration have been consistently in the predetermined range;
or evaluating for progressivity of glucose measurements by:
querying as to whether there at least P consecutive prior glucose measurements above range or below range; and
if the querying step indicates that at least P consecutive glucose measurements outside of the range have been stored, annunciating a message that progress has been made in which the most recent glucose measurement is back in the range after being outside the range over one of a time duration or after at least P consecutive prior glucose measurements.

2. The method of claim 1, in which the querying further comprises deciding whether at least one of an above range flag or below range flag has been stored for the period of D days and if the querying and deciding steps are affirmative or true, annunciating a message that progress has been made in which the most recent glucose measurement is back in the range after being outside the range over one of a time duration or after at least P consecutive prior glucose measurements.

3. The method of claim 1, in which the deciding step further comprises gleaning from prior glucose measurements of at least P consecutive prior glucose results that are either above the range or below the range during the time duration.

4. The method of claim 1, in which the first threshold comprises about 50 milligrams of glucose per deciliter of blood and the second threshold comprises about two to four times that of the first threshold.

5. The method of claim 1, in which the checking step further comprises:
storing in the memory, a plurality of glucose measurements measured prior to the most recent glucose measurement in the measuring step and indexing each of the glucose measurements with a time and date at which each of the measurements was taken;
if the most recent glucose measurement is one of a value generally equal to or above the second threshold then evaluating for a high trend; and
if the most recent glucose measurement is one of a value generally equal to or below the first threshold then evaluating for a low trend.

6. The method of claim 5, in which the evaluating for a high trend comprises:
defining a referential time interval N that includes a start time before or at generally the same as a time point at which the most recent glucose measurement was taken and an end time after or at generally the same as the time point of the day of the most recent measurement;
applying the referential time interval of N to each of prior D days so that the referential time interval N brackets a time point for each prior day that is generally the same as the time point of the most recent glucose measurement;
determining whether X number of prior BG measurements falls within the referential time interval N as applied to each of the prior D days;
assessing whether each of the at least X number of prior BG measurements is of a value that is generally equal to or above the second threshold; and
if the assessing step indicates that each of at least X glucose measurement is of a value generally equal to or higher than the second threshold, storing a first flag indicative of an above range trend for the duration of D days.

7. The method of claim 6, in which the storing step further comprises annunciating a message to indicate an above range pattern has been detected if at least one prior glucose measurement is above the second threshold on any T1 days of prior W days.

8. The method of claim 5, in which the evaluating for a low trend comprises:
defining a referential time interval N that includes a start time before or at generally the same as a time point at which the most recent glucose measurement was taken and an end time after or at generally the same as the time point of the day of the most recent measurement;
applying the referential time interval of N to each of prior D days so that the referential time interval N brackets a time point for each prior day that is generally the same as the time point of the most recent glucose measurement;
determining whether Y number of prior BG measurements falls within the referential time interval N as applied to each of the prior D days;
assessing whether each of the at least Y number of prior BG measurements is one of a value generally equal to or below the first threshold; and
if the assessing step indicates that at least Y glucose measurement is at or lower than the first threshold, storing a second flag indicative of a below range trend for the duration of D days.

9. The method of claim 8, in which the storing step further comprises annunciating a message to indicate a below range pattern has been detected if at least one prior glucose measurement is below the first threshold on any T2 days of prior W days.

10. The method of claim 8, in which the referential time interval comprises a first time interval N1 before the time point and a second time interval N2 after the time point referenced by the time point at which the most recent glucose measurement was taken.

11. The method of claim 8, in which the start time is generally the same as the time point of the most recent glucose measurement was taken and the end time is about N hours after the time point of the most recent glucose measurement.

12. The method of claim 8, in which the start time is N hours before the time point of the most recent glucose measurement and the end time is generally the same as the time point of the most recent glucose measurement.

13. The method of claim 8, in which N comprises any value from about zero to about 10, Z comprises any value from about one to about seven, X comprises any value from about one to about fourteen, Y comprises the same value as X, T1 comprises any value from 1 to about 14, T2 comprises any value from about 1 to about 14, P comprises from about 2 to 7, and D comprises about 2 to about 90.

14. The method of claim 13, in which N comprises about 3, Z comprises about 3, X comprises about 3, Y comprises about 3, T1 comprises about 2, T2 comprises about 1, P comprises 3, and D comprises about 7.

15. A method of notifying users of glucose trends with a diabetes management unit having a microprocessor coupled to a memory, the method comprising:
measuring with the microprocessor, a most recent glucose measurement of a user;
determining whether the most recent glucose measurement from the measuring step is within a range;
conducting, in the event the most recent glucose measurement is within the predetermined range, at least one of a consistency analysis and progressivity analysis for prior glucose measurements and the most recent glucose measurement; and
annunciating one of (a) a message to the user of a result of the consistency analysis in which the most recent glucose and the prior glucose measurements over a time duration have been consistently in the range or (b) a message to the user of a result of the progressivity analysis in which the most recent glucose measurement is back in the range after being outside the range over one of a time duration or after at least P consecutive prior glucose measurements.

16. The method of claim 15, in which the measuring comprises inserting a glucose test strip into a test strip port of the diabetes management unit and depositing a drop of blood onto the glucose test strip.

17. The method of claim 15, in which the determining comprises comparing the most recent glucose measurement with a first threshold and a second threshold of the range.

18. The method of claim 17, in which the comparing further comprises:
storing in the memory, a plurality of glucose measurements measured prior to the most recent glucose measurement in the measuring step and indexing each of the glucose measurements with a time and date at which each of the measurements was taken;
if the most recent glucose measurement is one of a value generally equal to or above the second threshold then evaluating for a high trend; and
if the most recent glucose measurement is one of a value generally equal to or below the first threshold then evaluating for a low trend.

19. The method of claim 17, in which the evaluating for a high trend comprises:
assessing whether at least one glucose measurement of the plurality of glucose measurements performed on previous D days within a time frame of X hours about a time of the day of the most recent glucose measurement, is higher than the second threshold; and
If the assessing step indicates that at least one glucose measurement is higher than the second threshold, storing a first flag indicative of an above range trend for the duration of D days.

20. The method of claim 18 in which the evaluating for a low trend comprises:
evaluating whether at least one glucose measurement of the plurality glucose measurements performed on Y of the previous D days within a time frame of N hours about a time of the day of the most recent glucose measurement, is lower than the first threshold; and
if the evaluating indicates that at least one glucose measurement is lower than the first threshold, storing a second flag indicative of a below range trend for the duration of D days.

21. The method of claim 16, in which the conducting comprises evaluating for consistency of prior glucose measurements by:
assessing for an absence of consistency message annunciated in prior D number of days,
determining whether at least one or more glucose measurements made for each of Z number of days within the period of prior D days;
obtaining a number of glucose measurements of the plurality of stored glucose measurements for D days that is within the range;
calculating whether the number is greater than a predetermined value; and
if the assessing, determining and calculating steps are affirmative, annunciating a message to the user of a result of the consistency analysis in which the most recent glucose and the prior glucose measurements over a time duration have been consistently in the predetermined range.

22. The method of claim 16, in which the conducting comprises evaluating for progressivity of glucose measurements by:
evaluating whether the most recent glucose measurement is within the predetermined range;
determining whether at least one of an above range flag or below range flag has been stored for the period of D days; and
annunciating a message that progress has been made in the event the determining step indicates that at least one of the above range flag and below range flag has been stored and the evaluating reflects that the most recent glucose measurement is within the predetermined range.

23. The method of claim 16, in which the conducting comprises evaluating for progressivity of glucose measurements by:
evaluating whether the most recent glucose measurement is within the predetermined range;
determining whether at least P consecutive prior glucose measurements have been outside of the range; and
annunciating a message that progress has been made in the event the determining step indicates that at least P consecutive prior glucose measurements have been outside of the range and the evaluating reflects that the most recent glucose measurement is within the predetermined range.

24. The method of claim 21, in the event that the assessing, determining, and calculating steps are negative or the evaluating, determining, and annunciating steps are negative, annunciating a message to continue with glucose measurements.

25. The method of claim 21, in which the annunciating comprises displaying an indicia representative of consistency or progressivity in glucose measurements.

26. The method of claim 24, in which the indicia comprises a green colored graphical symbol on a display of the diabetes management unit.

27. The method of claim 19, in which the storing step further comprises annunciating a message to indicate an above range pattern has been detected if at least one prior glucose measurement is above the second threshold on any T1 days of prior W days.

28. The method of claim 20, in which the storing step further comprises annunciating a message to indicate a below range pattern has been detected if at least one prior glucose measurement is below the first threshold on any T2 days of prior W days.

29. The method of claim 20, in which N comprises any value from about zero to about 10, Z comprises any value from about one to about seven, X comprises any value from about one to about fourteen, Y comprises the same value as X, T1 comprises any value from 1 to about 14, T2 comprises any value from about 1 to about 14, P comprises from about 2 to 7, and D comprises about 2 to about 90.

30. The method of claim 29, in which N comprises about 3, Z comprises about 3, X comprises about 3, Y comprises about 3, T1 comprises about 2, T2 comprises about 1, P comprises 3, and D comprises about 7.

31. A chronic disease management system comprising:
a biosensor unit that provides physiological data of a user; and
a chronic disease management unit comprising:
a microprocessor in communication with the biosensor unit to receive a plurality of physiological measurements reflective of a health condition of the user, the microprocessor being coupled to a memory; the microprocessor being configured to:
store the plurality of physiological measurements as collected from the biosensor;
determine whether a most recent physiological measurement is within a predetermined range;
evaluate (a) whether the plurality of physiological measurements including the most recent physiological measurement are consistently within the predetermined range over a time duration or (b) whether the most recent physiological measurement is within the predetermined range while prior plurality of physiological measurements have been out of the predetermined range during a time duration; and
annunciate a message indicating a result of evaluation (a) or (b) to the user.

32. The system of claim 31, in which the message of the result of the evaluation for consistency analysis comprises at least one of an:
(1) indication that a percentage of physiological measurements including the most recent physiological measurement in the last D days are in the predetermined range;
(2) indication that a number out of a total number of physiological measurements including the most recent measurement in the last D days are in the predetermined range; or
(3) indication that the user is doing well by having a percentage of the physiological measurements including the most recent physiological measurement in the last D days are in the predetermined range.

33. The system of claim 32, in which the message of the result of the evaluation for progressivity analysis comprises at least one of an:
(1) indication that the most recent physiological measurement is back in the predetermined range;
(2) indication that the most recent physiological measurement is back in the predetermined range after being a number of times out of the predetermined range; or
(3) indication that the user is back in the predetermined range after being out of the predetermined range.

34. The system of claim 33, in which the evaluation by the microprocessor for consistency of physiological measurements is by:
an assessment for an absence of consistency message annunciated in prior D number of days,
a determination of whether at least one or more physiological measurements made for each of Z number of days within the period of prior D days;
an estimation of a number of physiological measurements of the plurality of stored physiological measurements for D days that is within the predetermined range;
a calculation of whether the number is greater than a predetermined value; and
if the assessment, determination and calculation by the processor are affirmative, the processor is configured to annunciate a message to the user of a result of the consistency analysis in which the most recent physiological measurement and the prior physiological measurements over a time duration have been consistently in the predetermined range.

35. The system of claim 33, in which the evaluation by the microprocessor for progressivity of physiological measurements is by an evaluation of whether the most recent physiological measurement is within the predetermined range, a determination of whether at least P consecutive prior physiological measurements out of the predetermined range, and the processor is configured to annunciate a message that progress has been made by the user in the event the microprocessor determines that the determination indicates at least P consecutive prior P physiological measurements out of the predetermined range and the evaluation by the microprocessor reflects that the most recent physiological measurement is within the predetermined range.

36. The system of claim 35, in which the predetermined range comprises about 60 milligrams of glucose per deciliter of blood to about 180 milligrams of glucose per deciliter of blood, N comprises about 3, Z comprises about 3, X comprises about 3, Y comprises about 3, T1 comprises about 2, T2 comprises about 1, P comprises about 3, and D comprises about 7.
